(19) United States
(12) Patent Application Publication
(10) Pub. No.: US 2010/0086941 A1
(43) Pub. Date: Apr. 8, 2010

(54) Methods for determining aged based accumulation of senescent cells using senescence specific DNA damage markers

(76) Inventors: Guy R. Adami, Brookfield, IL (US); Suchismita Panda, Bolingbrook, IL (US)

Correspondence Address: MCCRACKEN & FRANK LLP, 311 S. WACKER DRIVE, SUITE 2500, CHICAGO, IL, 60606, US

(21) Appl. No.: 12/587,089

(22) Filed: Oct. 1, 2009

Related U.S. Application Data
(60) Provisional application No. 61/101,896, filed on Oct. 1, 2008.

(51) Int. Cl. G01N 33/53

(52) U.S. Cl. 435/7.1

(57) ABSTRACT

One disclosure provides a method for determining a senescence based disorder by detection of cells with senescence specific DNA damage markers which includes the step of providing a sample with one or more cells. It also includes the steps of identifying with immunodetection the presence of activated DNA damage response proteins that are shown to be activated with senescence and identifying with immunodetection the inactivation of DNA damage response proteins that are shown to be inactive in senescence.

### CROSS REFERENCE TO RELATED APPLICATIONS

This application claims the benefit of U.S. provisional application No. 61/101,896 filed Oct. 1, 2008.

### REFERENCE REGARDING FEDERALLY SPONSORED RESEARCH OR DEVELOPMENT

Funding for the invention was provided in part by grant R01 CA 85529-05 of the National Cancer Institute. The government has certain rights in this invention.

### SEQUENTIAL LISTING

Not applicable

### BACKGROUND OF THE INVENTION

1. Field of the Background

This present invention relates to cellular senescence, in particular, methods and kits to detect senescent cells that differentiates them from cells that show an active DNA damage response but are not truly senescent. This invention further relates to mechanisms of cellular senescence and, in particular, to the role of DNA repair and the DNA damage checkpoint pathways in the induction and maintenance of the senescent state.

2. Description of the Background

Cellular senescence is an irreversible block to cell cycle entry and cell proliferation that is defined by an inability of cells to enter S phase, or proliferate, even under ideal conditions (Cristofalo et al., 2004; Campisi, 2005). It has been shown to block primary cells from unlimited proliferation in vitro (Wright and Shay, 2002). One way that cellular senescence may contribute to the aging phenotype is if aging organs accumulate senescent cells that are altered with respect to normal function and lack the ability to respond to stress (including proliferation when required) (Holliday, 1995; Hornsby, 2001; Campisi, 2005). If enough of these cells are present, it might result in the reduced fitness seen with aging. A key component of this model is that substantial numbers of senescent cells should be present in tissues with aging, without, or prior to, pathology.

It is estimated that each mammalian cell may have thousands of molecular lesions such as DNA breaks per day (Molecular Biology of the Cell, p 963. WH Freeman: New York, N.Y. 5th ed). DNA damage response (DDR) is one method by which a cell can begin repairs of breaks in the DNA. This can take anywhere from hours to days. The DNA damage response is a quite common process and can cause a cell to be mistaken for a senescent cell.

Cell senescence was first systematically described approximately 30 years ago, in such publications as L. Hayflick, “The limited in vitro lifetime of human diploid cell strains,” Exp. Cell Res., 37:614 (1965); and Hayflick and Moorhead, “The serial cultivation of human diploid cell strains,” Exp. Cell. Res., 25:585 (1961). Despite the fact that cell senescence was first described long ago, our molecular understanding of cell senescence is still incomplete (U.S. Pat. No. 5,795,728).

Current methods to monitor senescence in individual cells have limitations. These include assays of the senescence-associated heterochromatin formation (SAHF) that occur in senescent cells visualized after DAPI staining. These areas of condensed chromatin typically consist of whole chromosomes with high concentrations of heterochromatin-associated proteins such as Histone H3 (tri methyl K9) and Hp1 alpha (Narita et al., 2003; Adams, 2007). Though these changes are well defined in vitro in certain human cells, they are by no means uniform even within a single cell type (Narita et al., 2003). Preliminary work suggests that SAHF occur in senescent murine cells, but care must be taken to differentiate them from the heterochromatin of satellite DNA repeats that occurs even in proliferating murine cells (Guenatri et al., 2004; Braig et al., 2005; Adams, 2007). The enrichment in cell cycle inhibitory proteins, such as p21 and p16, is also associated with senescence (Herbig and Sedivy, 2006; Collado et al., 2007). While overall p16 mRNA has been rigorously shown to increase with aging, there is less evidence that the enrichment of the protein can be used to identify individual senescent murine cells in vivo (Collado et al., 2007; Bartkova et al., 2005). The DNA damage response has been shown to be activated in senescent cells but it is not clear how to differentiate senescent cells from those with DNA damage that are not senescent. A method to circumvent this limitation is applicable to a subset of tissues and some mammals where telomere based senescence is dominant, such as primate skin fibroblasts (Jeyapalan et al 2007). SA β Gal, a lysosomal enzymatic activity increased in senescent cells in vitro kept under optimal growth conditions, is visualized as a blue peri-nuclear staining that correlates with cellular senescence when assayed at pH 6.0. Large numbers of SA β Gal positive cells are seen in vivo with disease (such as hepatitis, atherosclerosis, kidney fibrosis, carcinogenesis, prostatic hyperplasia) but the accumulations seen with old age are almost always minimal (Dimri et al., 1995; Melk et al., 2003; Cristofalo, 2005; Collado et al., 2007). While SA β Gal is not accepted as a marker of senescence under all conditions there is a good understanding of the assay's limitations especially in vitro (Cristofalo, 2005; Severino et al., 2000; Yang and Hu, 2005).

What is needed is a means to identify senescent cells present in a mixed cell population. Such a method should ideally permit the researcher to differentiate senescent from non-senescent quiescent, terminally differentiated, physiologically compromised cells or DNA damaged cells (U.S. Pat. No. 5,795,728). Methods that differentiate cells that enter senescence due to normal aging from those that temporarily appear senescent due to DNA damage are important tools to study these cells.

The disclosure allows for differentiation of senescent cells from cells that show an active DNA damage response but are not truly senescent.

### SUMMARY OF THE INVENTION

One embodiment of the present invention is a method for determining senescence based aging or disorder by detection of cells with senescence specific DNA damage markers in a sample comprising one or more cells that includes the steps of identifying with immunodetection the presence of activated DNA damage response proteins in the sample that are shown to be persistently activated in senescent cells and identifying with immunodetection the presence of insignificant activation of DNA damage response proteins in the sample that are shown not to be persistently activated in senescent cells.

Another embodiment is a method for determining senescence based aging or disorder in an organ by detection of cells with senescence specific DNA damage markers in a sample comprising one or more cells from the organ that includes the steps of identifying with immunodetection the presence of activated DNA damage response proteins in the sample that are shown to be persistently activated in senescent cells and identifying with immunodetection the presence of insignificant activation of DNA damage response proteins in the sample that are shown not to be persistently activated in senescent cells.

A further embodiment is a kit for differentiating senescent and DNA damaged cells that includes an immunodetection system for the presence of activated DNA damage response proteins in the sample that are shown to be persistently activated in senescent cells; and an immunodetection system for the presence of inactivation of DNA damage response proteins in the sample that are shown not to be persistently activated in senescent cells. The kit further includes cell preparation solutions; blocking agents; reagents; a buffer solution and; a fixing solution.

Other aspects and advantages will become apparent upon consideration of the following detailed description and the attached drawings, in which like elements are assigned like reference numerals.

### BRIEF DESCRIPTION OF THE DRAWINGS

FIG. 1A-B shows hepatocyte cells after exposure to a DNA damaging agent aminoazotoluene, (AAT) and how the cells look after different types of labeling and/or staining as well as nonexposed old mice. It also contains a graph of the percent of stained and/or labeled cells at different time periods after exposure to DNA damaging agent AAT as well as in controls and old mice.

FIG. 2 shows hepatocyte cells after exposure to DNA damaging agents (AAT) and mitomycin C and further shows how the cells look after different types of labeling and/or staining as well as a control group at different times.

FIG. 3A-B shows hepatocyte cells before and after exposure to a DNA damaging agent AAT and how the cells look after different types of labeling and/or staining at different times. It also contains a graph of the percent of stained and/or labeled cells at different time periods after exposure to DNA damaging agent AAT as well as a control group at different times.

FIG. 4A-C shows hepatocyte cells after exposure to a DNA damaging agent AAT or in nonexposed old mice and how the cells look after different types of labeling and/or staining.

FIG. 5A-F shows hepatocyte cells after exposure to a DNA damaging agent AAT or in nonexposed old mice and how the cells look after a hybrid SA β Gal assay as well as graphs showing the percentage of cells that were SA β Gal positive at various times after beginning the assay.

FIG. 6A-B shows accumulation of hepatocytes with ATM/ATR (p) S/QT DDR foci after exposure to a DNA damaging agent and with aging as well as graphs showing quantitation of ATM/ATR (p) S/TQ positive hepatocytes at various times after AAT exposure.

### DETAILED DESCRIPTION OF THE INVENTION

Normal, somatic cells generally divide for only a limited number of population doublings in vitro under standard tissue culture conditions until they senesce. When cell proliferation in a culture spontaneously ceases, most cells acquire an enlarged morphology and express a range of markers, some of which are also associated with cellular stress. Although senescent cells are unable to divide, they are nevertheless metabolically active and can be maintained in culture for long periods of time. Sub-optimal growth conditions can be responsible for the induction of growth arrest and the senescent phenotype (Tang, D. G. et al. Science 291, 868-71 (2001), Mathon, N. F. et al. Science 291, 872-5 (2001)). However, many other stresses including direct DNA damage, whether random or due to telomere shortening, can result in senescence even for cells kept in standard culture conditions. While there are many ways to initiate the senescence process, the DNA damage response (DDR) plays a role in the senescence process even when DNA damage is not the initiating event (von Zglinicki et al., Mech Aging Dev 126, 111-7.(2005).

The DNA damage response is directly induced in cells due to DNA DSBS (double strand breaks), the primary cytotoxic lesions caused by ionising radiation (IR) and radio-mimetic drugs. Cells react to DSBS by mounting a range of responses, including the activation of DNA repair mechanisms and the triggering of checkpoint events whose primary function is to halt or slow cell cycle progression until the DNA damage has been removed (Shiloh, Y. Nature Reviews Cancer 3, 155-68 (2003), Nyberg, K. A. et al Annu Rev Genet 36, 617-56 (2002), Khanna & Jackson Nat. Genet 27 247-254 (2001)). Treatment of human cells with IR leads to the rapid activation of the DNA-damage transducer protein kinases ATM and ATR. These kinases then phosphorylate and activate a series of downstream targets, including the effector protein kinases CHK1 and CHK2, and the checkpoint mediator proteins 53BP1 and MDC1. Phosphorylation of a set of ATM/ATR target proteins results in the formation of microscopic foci consisting of large accumulations of proteins such as 53BP1, H2AX gamma, and ATM itself near the site of the break. The formation of DDR foci at sites of DNA DSBs is characteristic feature of the checkpoint response (Goldberg, M. et al. Nature 421, 952-6 (2003)). Additional regulatory proteins are activated, such as Chk1, Chk2, and p53, which can be found throughout the nucleus. The DDR proteins serve to sense the break, amplify the DNA damage signal, and formulate a cellular response (repair, cell death, and/or senescence). In laboratory animals in a controlled environment, with the exception of gamma H2AX, the activation of DDR proteins is seen only at very low levels in tissues in young rodents and primates (Sedelnikova et al., 2004; Jeyapalan et al., 2007).

The DDR is a fundamental part of senescence, whether induced spontaneously by a gradual replication dependent telomere attrition, or by entry into a “stress induced senescence-like arrest” due to oxidative damage, direct DNA damage, or inappropriate oncogene activation (d'Adda di Fagagna et al., 2003; Takai et al., 2003; Sedelnikova et al., 2004; von Zglinicki et al., 2005; Herbig and Sedivy, 2006; Collado et al., 2007). The DDR is thought to be required for initiation and maintenance of cellular senescence An examination of cells in primates has shown an age-related increase in the DDR in skin fiboblasts, but not muscle myocytes, exemplified by the presence of DNA damage foci (Jeyapalan et al., 2007). Cells with similar markers increase early in the neoplastic process (Bartkova et al., 2005; Chen et al., 2005; Gorgoulis et al., 2005; Jeyapalan et al., 2007). Potentially senescent cells with DNA damage foci occur in large numbers in vivo with many diseases, but, with the exception of mammalian dermis, there is little evidence for that with normal aging.

There is wide consensus that senescent cells accumulate in vivo with pathology, but there is much less evidence that they accumulate above extremely low levels with normal aging (Dimri et al., 1995; Melk et al., 2003; Collado et al., 2007). The liver is no exception. That SA β Gal positive cells accumulate with aging in the unperturbed healthy livers of normal (and in the case of mice, wild type) old subjects is not supported by most studies (Krishnamurthy et al., 2004), though they certainly accumulate with diseases such as cancer and cirrhosis. It is not clear how readily hepatocytes do senesce. While DNA damage signals in the form of telomere deprotection induce senescence in proliferating liver hepatocytes, they may lack this property in nonproliferating adult liver hepatocytes (Lechel et al., 2005; Lazzerini Denchi et al., 2006). Changes in the liver and liver hepatocytes, even without the presence of overt disease, have long been known to occur with old age. These changes include decreases in liver size and numbers of hepatocytes, with increased average ploidy of the remaining hepatocytes (Gupta, 2000). While the turnover of liver hepatocytes is normally slow in the adult, it can become rapid in response to injury. In old organisms, the ability of the liver to regenerate is reduced; the process takes longer and involves fewer cells (Stocker and Heine, 1971; Gupta, 2000). However, major changes also occur in hepatocytes as early as young adulthood, such as the first accumulation of polyploid cells and the loss of extensive replicative capacity of the cells when plated in vitro. To characterize cells with an activated DDR in this organ, we used a single exposure to juvenile mice of AAT, a DNA alkylating agent that is activated in liver hepatocytes or to a second genotoxin mitomycin C (Zimmerman, 1999).

If cellular senescence is a long-lived state of DDR activation, and senescent cells can accumulate in vivo in normal tissue, then cells with an activated DDR should be detectable long after artificial induction of senescence. Data has shown that cells with an activated ATM protein kinase and DNA damage foci can occur with advanced age, but at least certain components of a true active DDR state are lacking. That is supported by the observation that 1 week after mitomycin C exposure cells with a robust DNA damage response are present, but 7 weeks after exposure, when the mitomycin C has long ago been cleared from the body, senescent cells are present but they lack activation of the complete DDR. By immunostaining for individual activated or inactivated DDR proteins the status of a cell can be proven.

Senescence associated disorders include any disorder which is fully or partially mediated by the induction or maintenance of a non-proliferating or senescent state in a cell or a population of cells in an individual. Examples include age related tissue or organ decline which can lack visible indication of pathology, or overt pathology such as coronary disease, impaired wound healing, inflammation, infection, neoplasia, immune dysfunction, Alzheimer's disease, liver cirrhosis, or immunosenescence. A method of identifying a senescence associated disorder as described herein may include determining the activity of a DNA damage response pathway polypeptide.

Activities which may be determined may include kinases and other polypeptides. These activities may be determined using conventional techniques. In some preferred embodiments, for example when the polypeptide is ATM, ATR, Chk1 or Chk2, the kinase activity of the polypeptide may be determined.

The phosphorylation of a DNA damage response pathway polypeptides may be indicative of its activated state. Activity may also therefore be determined by determining the phosphorylation of a DNA damage response pathway polypeptide. DNA damage response pathway polypeptides which are activated by phosphorylation include but are not limited to CHK1, CHK2, NBS1, MRC1 and ATM. Additional DDR proteins include ATRIP, BRCA1, RAD50, MRE11, CDC25C, 14-3-3.sigma., CDK2/cyclin E, CDK2/cyclin B1 53BP1, MDC1, histone variant H2AX, SMC1, RAD17, RAD1, RAD9, and HUS1.

Activation of the DNA damage response pathway may be determined by any convenient method. For example, activation of the pathway may be determined by determining the activation of one or more components of the pathway. In some embodiments, for example, the kinase activity of one or more DNA damage checkpoint kinases, such as ATM, ATR, CHK1 or CHK2 may be determined.

In other embodiments, activation of the DNA damage response pathway activity may be determined by determining the phosphorylation of one or more DNA damage checkpoint polypeptides. Examples of DNA damage response polypeptides which are activated by phosphorylation include ATRIP, CHK1, CHK2, BRCA1, NBS1, RAD50, MRE11, CDC25C, 14-3-3.sigma., CDK2/cyclin E, CDK2/cyclin B1 53BP1, MDC1, histone variant H2AX, SMC1, RAD17, RAD1, RAD9, HUS1 and MRC1.

Phosphorylation may be determined by any suitable method known to those skilled in the art. It may be detected by methods employing radiolabeled ATP and optionally a scintillant. By way of example, phosphorylation of a protein may be detected by capturing it on a solid substrate using an antibody or other specific binding molecule directed against the protein and immobilised to the substrate, the substrate being impregnated with a scintillant--such as in a standard scintillation proximity assay. Phosphorylation is then determined via measurement of the incorporation of radioactive phosphate.

Phosphate incorporation may also be determined by precipitation with acid, such as trichloroacetic acid, and collection of the precipitate on a nitrocellulose filter paper, followed by measurement of incorporation of radiolabeled phosphate.

Phosphorylation may also be detected by methods such as immunostaining or employing an antibody or other binding molecule which binds the phosphorylated polypeptide with a different affinity to unphosphorylated polypeptide. Such antibodies may be obtained by means of any standard technique as discussed elsewhere herein. Binding of a binding molecule which discriminates between the phosphorylated and non-phosphorylated form of a polypeptide may be assessed using any technique available to those skilled in the art, examples of which are discussed elsewhere herein. The usage of antibodies may be either polyclonal or monoclonal to detect polypeptides. Additionally new and theoretical nano-based synthetic detection systems could achieve the same results.

Certain DDR proteins are persistently activated in senescent cells while other proteins are transiently or not persistently activated in senescent cells. The ability to show whether certain kinases and proteins at the foci are persistently activated allows the determination of the cell being senescent or in DNA damage response. For example, activation of 53BP1 and a subset of ATM targets so that these proteins are maintained in DDR foci occurs in both senescent cells and those undergoing DNA damage repair. Activation of DNA damage response proteins P53, Chk2 which can be marked by the phosphorylation, though they are not typically in foci, only occurs in cells undergoing DNA damage response, not senescent cells and therefore is not persistently activated in senescent cells. By indicating the activation of 53BP1 and a subset of ATM targets that are maintained in DDR foci in senescent cells and the inactivation of proteins P53 and Chk2, a cell can be determined to be senescent.

The following polypeptides are persistently active in senescent cells and are also present in cells undergoing DNA damage response: 53BP1, additional ATM/ATR targets found in DDR foci such as NBS1.

Senescent cells do not show an activation of the following DDR polypeptides: CHK2, p53, or are greatly reduced in activity ATM, all of which are highly activated by DNA damage. These proteins are not persistently activated in senescent cells.

A test kit could be easily created using immunostaining with antibodies for 53BP1, Chk2, P53, and ATM. One embodiment where this assay can used is to test effectiveness on therapy to reduce senescent cells.

One method of testing for a cell to be senescent involves immunostaining with flurochromes/fluorophores that are tuned to two different wave lengths so that they emit light at two different colors. Immunostaining could occur for a protein such as 53BP1 which is active in both senescence and DDR with one antibody. Immunostaining could then occur for active CHK2 which would not be present in a senescent cell with the second colored antibody. An emission of two colors would signal DDR while only one would signal senescence. This could also be done with a single colored antibody using two tissue sections from the same sample and one antibody. One embodiment where this assay can be used is to test effectiveness on therapy to reduce senescent cells.

This method will be useful in validating the results of clinical trials or a compound that would treat senescent associated disorders. With the recent discovery of biomarkers and the polypeptides involved in regulating cell senescence, treatments for cell senescence disorders have been proposed. Methods and means for the treatment of disorders associated with cellular senescence have been proposed.(US 2007/0099186A1) One difficulty associated with these treatments will be validating the successful results. Cells in a state of senescence appear similar to cells undergoing DNA damage repair. Cells merely undergoing short term DDR will eventually return to a normal state. If a group of cells are treated with a proposed senescence disorder treatment and then some cells leave an assumed state of senescence it would be difficult know if those cells were truly senescent or those merely undergoing DNA damage repair. In addition, this assay can be used to test effectiveness of therapy to reduce senescent cells.

It is further contemplated that the method of the present disclosure will provide a method designed to measure fitness of tissues and organs used for transplantation. For example, it is contemplated that the methods of the present invention will serve to identify senescent cells in tissues to be transplanted. One embodiment would be a method to determine aging of the liver based on immunofluorescent detection and counting of cells with aggregates of specific nuclear DNA damage focus proteins.

### Experimenatal Overview

The following examples serve to illustrate certain preferred embodiments and aspects of the present disclosures and are not to be construed as limiting the scope thereof.

After experimental induction of cellular senescence in the livers of juvenile mice, there was robust expression of DDR markers in hepatocytes at 1 week; however, by 7 weeks, activation of ATM/ATR kinase targets was limited, although cells with 53BP1 foci were present. An analysis of hepatocytes of aged, 22-month-old mice, not experimentally exposed to genotoxins, showed limited activation of some ATM/ATR targets, though high numbers of cells with foci with a subset of DNA damage response proteins were found, similar to that seen many weeks after artificial senescence induction in young mice.

Exposure to a DNA damaging agent had increased numbers of cells that showed a series of reproducible changes in DNA damage protein. At 3 and 7 weeks after exposure to either genotoxin, most positive nuclei had only a single prominent DNA damage focus. By 3 weeks, we saw the loss of DNA damage markers phospho Chk2 (T68), phospho p53 (S15), and by 7 weeks on average there was a reduction in the level of phospho ATM to near undetectable levels. Finally there was a decrease in ATM/ATR pS/TQ signal, which is specific for a subset of ATM/ATR targets including phospho NBS1 and CHK2 but it was still easily detectable.

In situ studies on tissue sections revealed that 1 week after induction of hepatocyte senescence, many DNA damage/senescence markers were enriched. By 7 weeks posttreatment, many cells still contained 53BP1/phospho ATM foci, though on average the foci were reduced in intensity for the phospho ATM and an additional subset of activated ATM/ATR substrates, and the cells lacked activated Chk2 (T68) and p53 (S15). While the ATM/ATR pS/TQ signal was reduced it was easy to detect, while the activation of the other proteins was undetectable or nearly undetectable. We saw similar reductions in these ATM/ATR targets 7 weeks after a single exposure to a second DNA damaging agent, mitomycin C. Finally, in old adult (22-month-old mice), but not in young adult (4-month-old mice), high numbers of cells with the same subset of enriched DDR markers were found. This indicated that potentially senescent cells accumulated in the murine liver with aging but were enriched for only a subset of DDR-linked proteins or protein modifications.

### Example 1

Eight to eleven-day-old F1 mice from C57BL/6, obtained from the National Institute of Aging, Aged Rodent Colonies (Bethesda, Md.), were injected intraperitoneally with AAT (Sigma-Aldrich; St. Louis, Mo.) at 2 mg/kg body weight in corn oil. Mitomycin C (Sigma-Aldrich) was injected under a similar protocol at 2.5 mg/kg body weight in PBS. Control gender-matched litter mates were injected with corn oil or PBS. The pharmacokinetics for clearance of AAT are not known with certainty, while mitomycin C and its active metabolites are cleared with a half life of under 1 h (Dorr, 1988). Female C57BL/6 mice (4- and 22-month-old) were allowed to acclimate at least 1 week prior to usage. All procedures were done within the guidelines of the Animal Research Committee at the

University of Illinois at Chicago and the “Principles of laboratory animal care” (NIH publication No. 86-23, revised 1985).

To detect DNA damage foci, frozen 5-micron liver sections were rapidly transferred to slides after sectioning, then immediately fixed in 2% paraformaldehyde PBS for 20 min, and then prepared following standard protocols. Antibodies were used at the following dilutions, anti-phospho p53 (S15) and anti ATM/ATR phosphorylated consensus target sequence (p)S/TQ (Cell Signaling; Danvers, Mass.: 1:50 and 1:200 (v/v)), anti-53BP-1 (two separate antibodies raised against separate regions of the protein (Novus Biologicals; Littleton, Colo.; 100-305; 1:2,000; and 100-304; 1:1000) and anti-phospho Chk2 (T68) (Abcam; Cambridge, UK: 1:200), all rabbit antibodies. Anti-(p)S/TQ reacts with a large group of putative ATM/ATR targets to different degrees, showing strong affinity to CHK2 and NBS1 and weak affinity to phospho-ATM (S1981). The specificity was verified by blockage with oligopeptides bearing the antigenic sequences. Antiphospho ATM (S1981) (Rockland Immunochemicals, Gilbertsville, Pa.; 1:800) was a mouse antibody. Anti-rabbit and anti-mouse secondary antibodies conjugated to Cy-3 were obtained from Jackson Immunoresearch (West Grove, Pa.). Prior to mouse primary antibody application, tissues were first blocked with 1% goat anti-mouse FAB (Jackson Immunoresearch) followed by incubation with 4% BSA. Slides were counterstained with 0.2 ug/ml 4′,6-diamidino-2-phenylindole (DAPI) for 2 min prior to mounting in mowiol (Calbiochem; San Diego, Calif.) with 2% 1,4- diazobicyclo[2,2,2]octane (Sigma-Aldrich). Fluorescent 400× images were typically collected immediately after the assay. Foci-associated nuclei and total nuclei were counted using Image Pro Plus software (MediaCybernetics, Bethesda, Md.). Three to four mice were used for each time point and 300-500 nuclei were counted for each subject. Relative fluorescence intensity of foci in images was quantitated using the density measurement of the Image Pro Plus software with subtraction of local background levels. Four to seven mice were studied per time point with 8 to 18 53BP1/phospho ATM foci scored per mouse. Minimally exposed images were used. The student's t-test was used for statistical comparisons for these and other experiments. For double labeling with anti-phospho ATM (S1981) and anti-53BP1 antibodies, sections were prepared as described above, first labeling with the anti-phospho-ATM (S1981) and Cy-3 secondary antibody as above, then incubated with 4% BSA, followed by anti-53BP1 and then by Alexa 488 conjugated goat anti-rabbit antibody (Invitrogen; Carlsbad, Calif.; 1:200). All secondary antibodies were pre-adsorbed to reduce cross-reactivity as verified by negative control experiments.

### Detailed Explanation of Figures

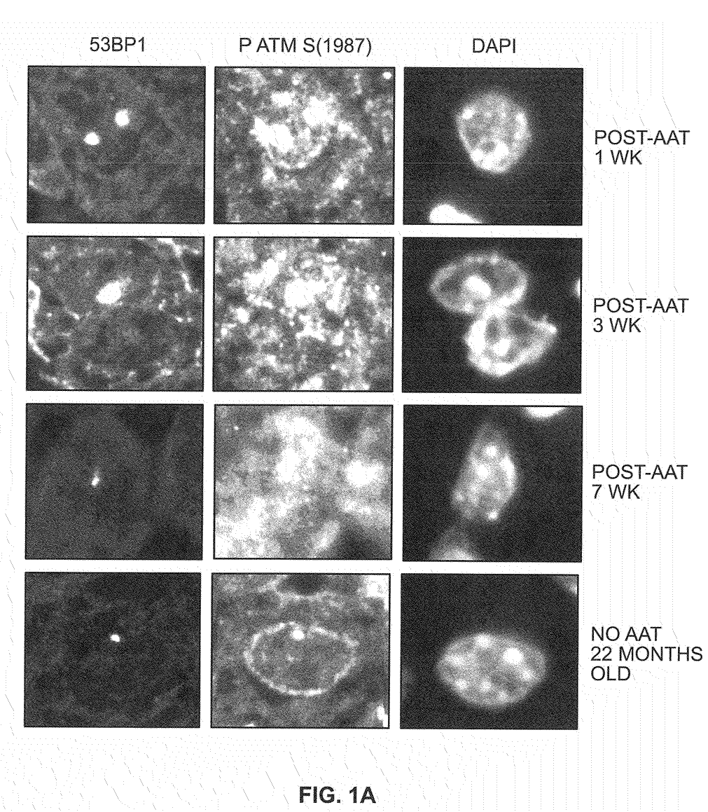

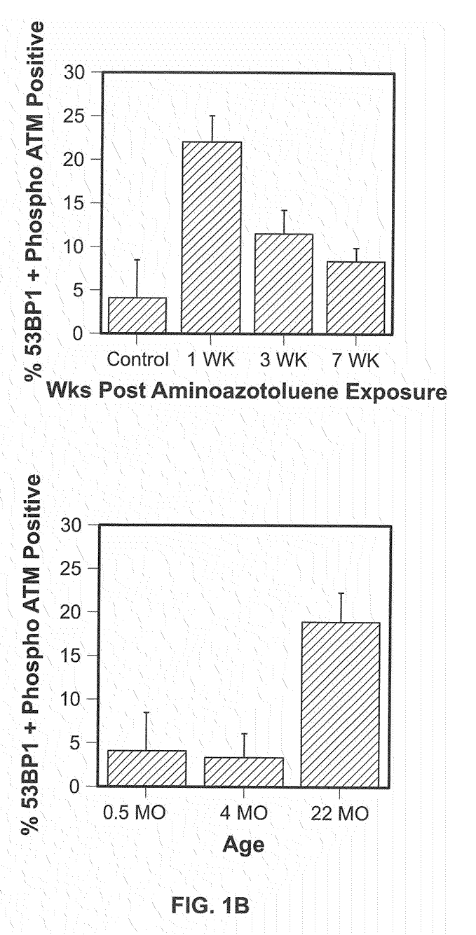

FIG. 1A-B FIG. 1A Accumulation of hepatocytes with DDR foci after exposure to a DNA damaging agent and with aging. Mice were exposed to a single injection of AAT, and at various time points tissue sections simultaneously immunostained for 53BP1 and phospho ATM (S1981). Mice at 22 months of age similarly revealed the presence of co-localized 53BP1/ATM1981 foci. Nuclear DNA was counterstained with DAPI. FIG. 1B Accumulation of hepatocytes with DDR foci after exposure to a DNA damaging agent and with aging. Quantitation of hepatocytes positive for 53BP1/phospho ATM co-localized foci at various times after AAT exposure and with advanced age, 3-6 mice at each time point. Control mice injected with vehicle and then assayed 1, 3 and 7weeks later all showed similar low levels of foci. At least 400 nuclei were scored for each animal.

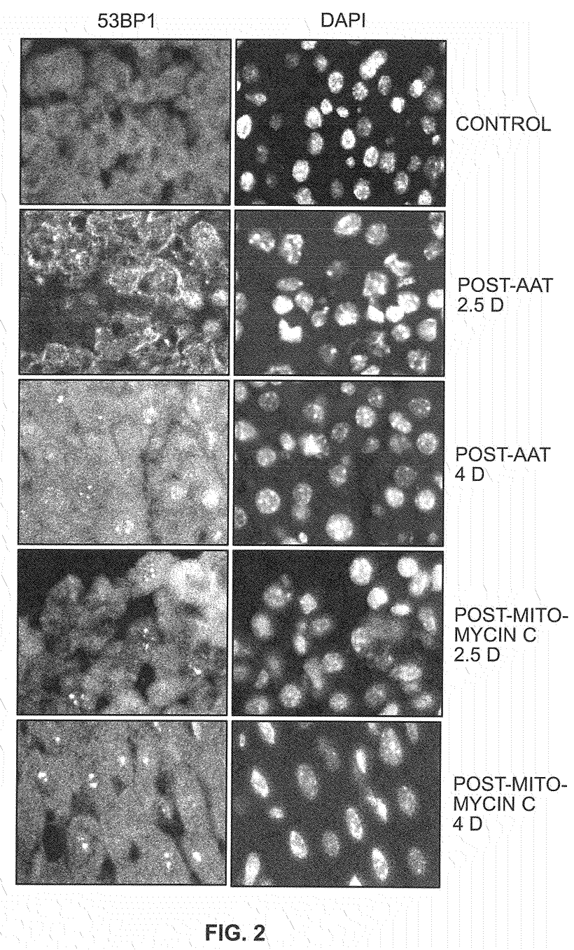

FIG. 2 Appearance of 53BP1 foci by 2.5 days after AAT or mitomycin C exposure. Immunolocalization revealed 53BP1 foci in hepatocytes of frozen liver sections. Multiple foci per nucleus were typically found at these early times. At the 2.5 day AAT time point nonspecific staining partially obscured the foci. Three mice were tested for all time points. Nuclei were counterstained with DAPI.

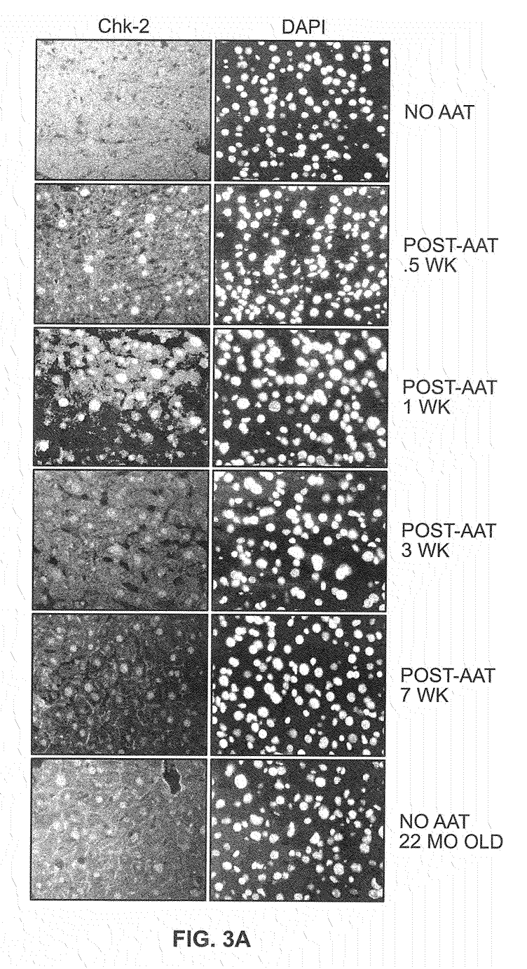

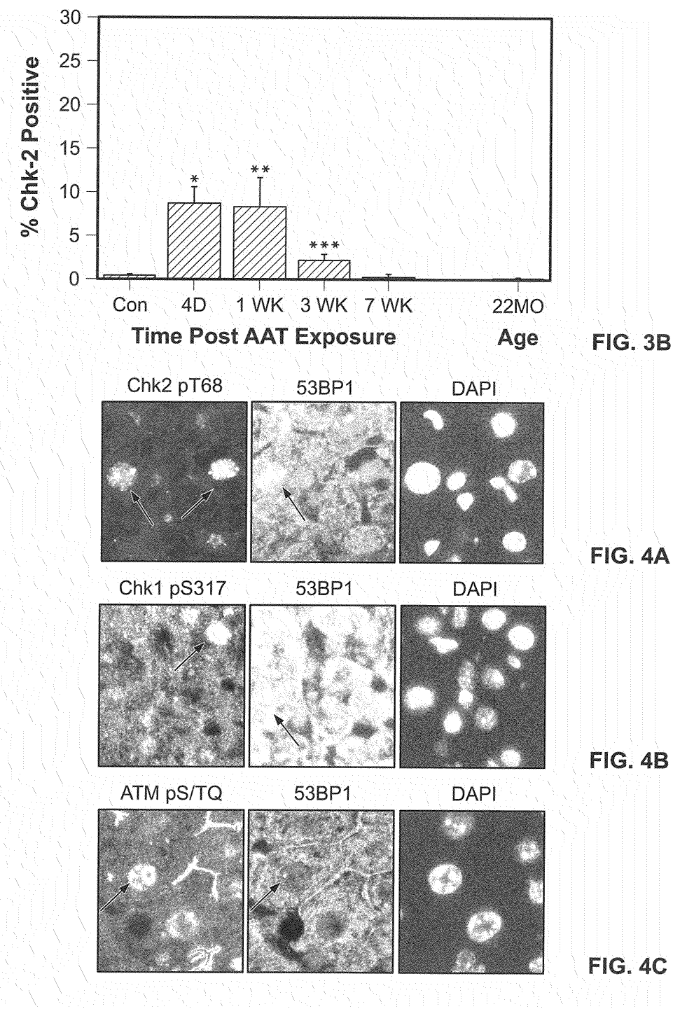

FIG. 3A-B Chk2 activation is greatly reduced long times after AAT exposure and is undetectable in 22-month-old mice. FIG. 3A At various times after AAT exposure tissue sections from liver were immunostained for phospho Chk2 (T68) nuclear staining. The control with no AAT was obtained 1 week after injection with vehicle. Controls from mice 0.5, 3 and 7 weeks after injection gave similar negative results. FIG. 3B Quantitation of phospho Chk2 positive nuclei revealed high levels of nuclear staining 0.5 and 1 week after AAT exposure. Over 500 nuclei were scored for each animal and 3-5 animals were tested for each time point. *P<0.0001; **P<0.0011; ***P<0.0019 by t-test.

FIG. 4A-C Co-localization of nuclear DDR proteins after AAT exposure and with aging. FIG. 4A One week after AAT exposure, immunofluorescence of frozen liver thin sections revealed co-localization of: phospho Chk2 and 53BP1 nuclear staining. FIG. 4B The same tissues showed lack of co-localization of phospho Chk1 and 53BP1 positive nuclear staining. FIG. 4C In 22-month-old mice ATM/ATR (p) S/TQ foci were found to typically co-localize with 53BP1 foci. Phospho Chk2 and ATM (p) S/TQ were induced by AAT exposure and/or age, but phospho Chk1 was not. Nuclei were counterstained with DAPI. For the above experiments we used anti 53BP1 (Novus Biologicals) which was direct labeled using the Alexa Fluor 488 monoclonal antibody labeling kit (Invitrogen) according to the manufacturer's instructions. Frozen sections were prepared as described in the experimental procedures, and then incubated with 4% BSA followed by anti-53BP1-Alexa Fluor 488 conjugated primary antibody. To block the sections they were then incubated with monovalent Fab fragment of donkey anti-rabbit IgG (H+L) (Jackson ImmunoResearch) followed by the second primary antibody and then anti-rabbit secondary body conjugated to Cy3.

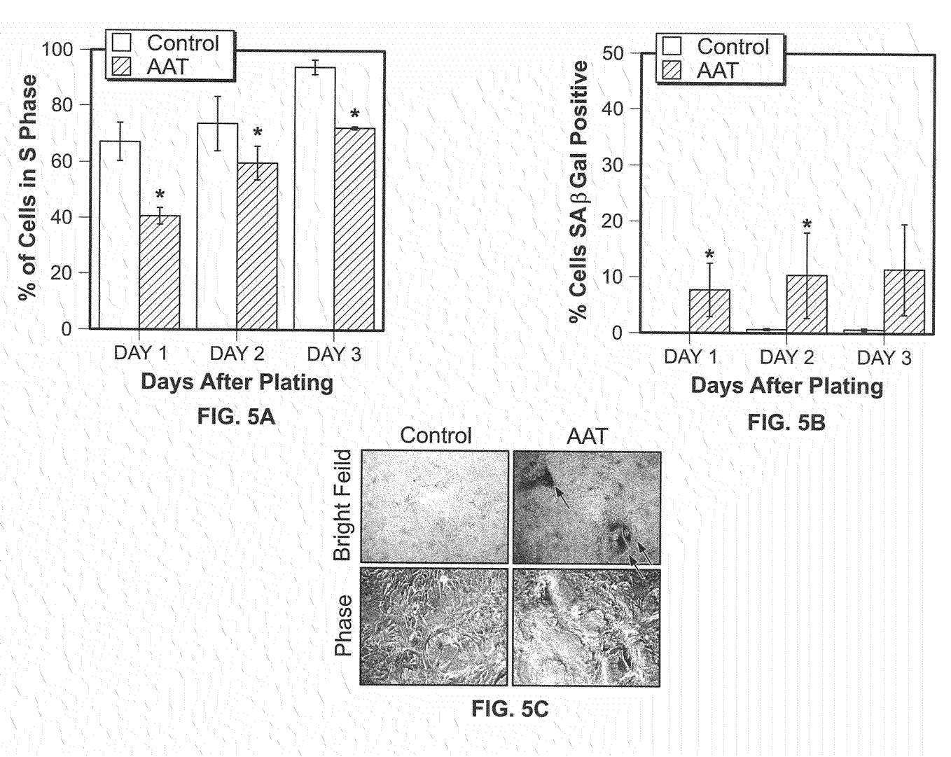

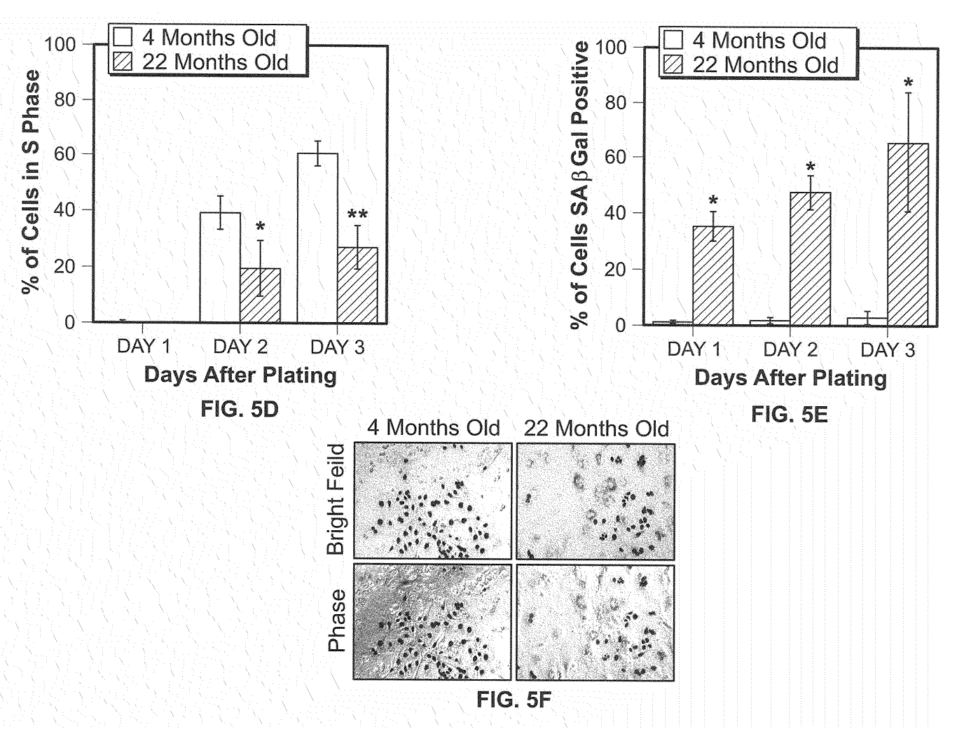

FIG. 5A-F Hepatocytes isolated from 22-month-old adult mice, but not 4-month-old young adult mice, are defective for S phase entry and positive for SA β Gal, as are hepatocytes from AAT exposed mice. FIG. 5A Collagenase perfusion was used to isolate hepatocytes from juvenile mice exposed 3 weeks previously to AAT. Twenty-four hours prior to fixation, cells were incubated with [3H]thymidine to allow the quantitation of the S phase index after processing for autoradiography. Shown are the means plus the (standard deviations) S.D. *P<0.002 versus the controls. FIG. 5B Cells isolated as described above from mice exposed, or not exposed, to AAT were subjected to the SA-B-Gal assay after fixation. At least 100 cells were counted on each plate from 3 to 5 control mice and 5 to 6 AAT exposed mice depending on the time point. Shown are the means plus S.D. *P<0.05 versus the controls. FIG. 5C Shown is a representative bright field image of primary hepatocytes 3 days after culture. Arrows indicate the blue, peri-nuclear stained, SA β Gal-positive cells. To reduce background [3H]thymidine detection was not done in this experiment. The phase contrast image below allows better visualization of the cells, original magnification 100×. FIG. 5D Hepatocytes isolated from adult 4-month-old and 22-month-old were cultured and assayed for S phase index as described. Shown are means plus S.D. There is a lag in S phase entry in cells from these adult mice. *P<0.02 and **P<0.004. FIG. 5E Hepatocytes from young adult and aged adult mice were cultured as described and then subjected to the SA β Gal assay. Shown are means plus S.D. *P<0.015 versus the respective controls. FIG. 5F Representative image of cells simultaneously assayed for [3H]thymidine labeling of nuclei, indicative of S phase transit, and SA β Gal. Cells from 4- and 22-month-old mice were plated 3 days prior to the assay. Original magnification 100×.

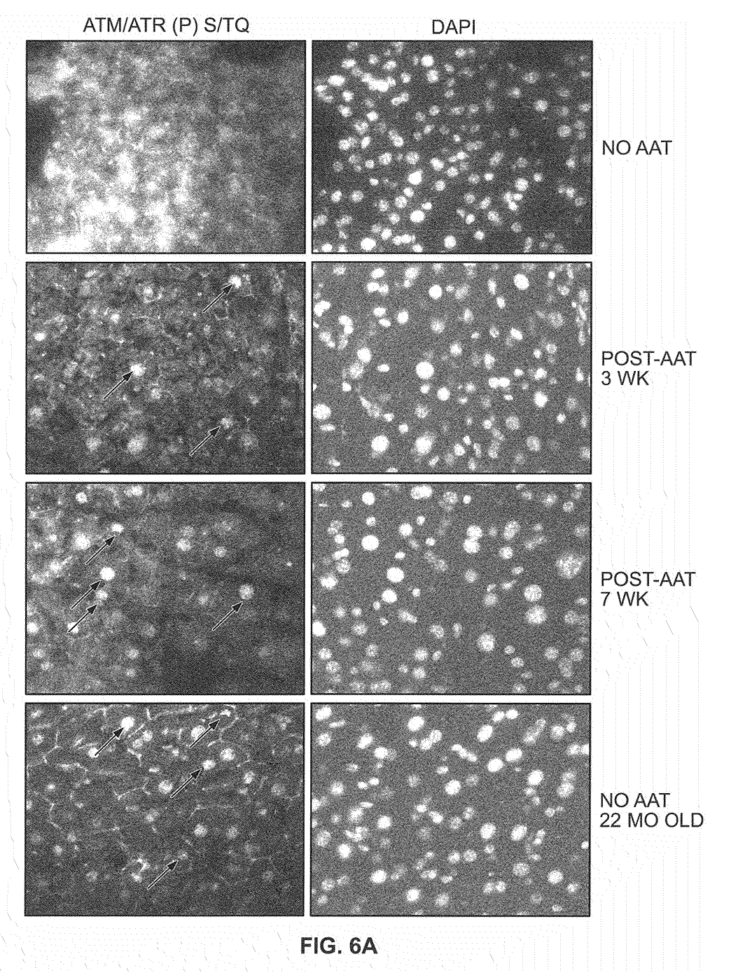

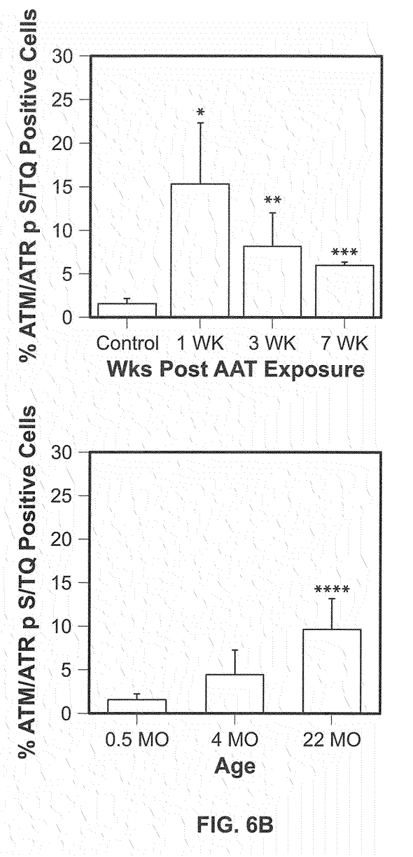

FIG. 6A-B. Accumulation of hepatocytes with ATM/ATR (p) S/QT DDR foci after exposure to a DNA damaging agent and with aging. FIG. 6A At 22 months of age, and at various times after AAT exposure, frozen sections from liver were immunostained for activated ATM/ATR (p) S/TQ substrate proteins. Arrows indicate sites of foci. Nuclei were counterstained with DAPI. FIG. 6B Quantitation of ATM/ATR (p) S/TQ positive hepatocytes at various times after AAT exposure and with advanced age, 3-6 mice at each time point. * P<0.025, ** P<0.033, *** P<0.0070, **** P<0.0090 by t-test. Control mice injected with vehicle and then assayed 1, 3 and 7 weeks later all showed similar low levels of foci. At least 400 nuclei were scored for each animal.

### Example 2

In this experiment activated 53BP1 and ATM, a subset of the ATM proteins that occur in foci, are shown to be present in both senescent and DDR cells.

To identify hepatocytes in vivo with long-term changes after a transient exposure to DNA damaging agents, young mice were given a single exposure to the DNA damaging agent, AAT, an azo compound that is activated in liver hepatocytes to become an alkylating agent that can induce cellular senescence in vitro (Zimmerman, 1999). Frozen liver tissue sections contained nuclear DNA damage foci at the earliest time point, 2.5 days post-exposure (FIG. 2). As shown, simultaneous detection of 53BP1 and phospho ATM after AAT exposure revealed elevated levels of nuclear foci containing both these proteins at 1 and 3 weeks after genotoxin exposure (FIGS. 1A and B). By 7 weeks after AAT exposure cells with 53BP1/phospho ATM foci were still present at higher levels than in the unexposed control mice (FIG. 1B). However, in time, we note 2 specific changes in the 53BP1/phospho ATM foci: first, prior to the 3-week time point many more nuclei with multiple foci were present; second, by 7 weeks after exposure to AAT, or to mitomycin C, a second DNA damaging agent, there were significant decreases in the signal strength of phospho ATM (FIG. 1A and Table 1) to levels that were so low they were difficult to detect or accurately quantitate (Tamura et al., 1992). This also occurred when ATM immunostaining was done singly without 53BP1 immunodetection (data not shown). The experiment with the second genotoxin, mitomycin C, was important because unlike AAT, the drug and its active metabolites have been shown to rapidly disappear from the body (Doff, 1988). At 2 or 3 days post exposure to mitomycin C, long-term effects on cells are observed, not responses to new chemical damage.

Next, this assay was used on liver sections from different aged mice unexposed to the DNA damaging agents. In 22-, but not 4-, month-old mice, there were high numbers of DNA damage foci containing co-localized 53BP1 and activated ATM, indicating the presence of substantial numbers of potentially senescent cells (FIG. 1B). Notably, these DNA damage foci were characterized by low signal strength for phospho ATM, similar to those observed in mice 7 weeks after AAT exposure (Table 1).

TABLE 1
Relative immunofluorescent intensity of
foci for ATM/ATR p (S/TQ) and ATM p (SI987)

| | ATM/ATR p (S/TQ) | ATM p (S1987) |
|---|---|---|
| 3 Wk post-AAT | 100 ± 20.0 | 100 ± 4.50 |
| 7 Wk post-AAT | 50 ± 17.5^{A} | 50 ± 9.80^{D} |
| 3-M-old (no AAT) | ND | 87.6 ± 34^{E} |
| 22-M-old (no AAT) | 50 ± 5.96^{B} | 55.4 ± 5.92^{F} |
| 3 Wk post-mitomycin C | 100 ± 10.1 | 100 ± 15.9 |
| 7 Wk post-mitomycin C | 69.6 ± 12.1^{C} | 57.4 ± 13.0^{C} |

^{A}3 Wk AAT versus 7 Wk AAT.P < 0.0001.
^{B}3 Wk AAT versus 22 M.P < 0.0001.
^{C}3 Wk MMC versus 7 Wk MMC, P < 0.0112.
^{D}3 Wk AAT versus 7 Wk AAT, P < 0.0171.
^{E}3 M vs.. 22 M, P < 0.019.
^{F}3 Wk AAT versus 22 M, P < 0.0047.
^{G}3 Wk MMC versus 7 wk MMC, P < 0.018.

### Example 3

DNA damage repair proteins such as Chk2 that are active in DDR are not active during senesce. An assay for protein activation by ATM/ATR kinases and downstream kinases, including p53 (S15), Chk2 (T68), and a generic ATM/ATR phosphorylated target sequence (ATM/ATR (p) S/TQ) was run. We saw activation of all of these with DNA damage in the AAT exposed mice (FIG. 3, FIG. 4, FIG. 5 and FIG. 6); however, except for ATM/ATR (p) S/TQ, all were activated only transiently, showing baseline or near baseline levels by 3-7 weeks post-AAT exposure. Mitomycin C exposure caused similar changes (Table 1). Focus proteins with the ATM/ATR (p) S/TQ sequence were, after exposure to either genotoxin, activated even 7 weeks after senescence induction but there was a reduction in signal intensity (Table 1). This antibody would detect a subset of the large group of activated ATM/ATR targets (Matsuoka et al., 2007), including NBS1 and Chk2, but it has minimal affinity for activated ATM itself (S. Manning, personal communication). When young and old mice were assayed, 22-month-old mice again showed levels of DDR proteins similar to those seen 7 weeks post-AAT exposure in young mice. There was no enrichment of activated phospho p53 (S15) or Chk2 (T68), while phospho ATM (S1981) positive cells were detected but the foci were of low intensity (FIG. 3, Table 1, data not shown). To verify that that there was an accumulation of senescent cells after AAT exposure and with aging in mice we used an independent method to test for senescent cells. A hybrid SA beta Gal assay was developed that determines the number of senescent cells in a population of hepatocytes isolated and placed in primary culture from mouse liver.(Fig. 5)

Numerous modifications will be apparent to those skilled in the art in view of the foregoing description. Accordingly, this description is to be construed as illustrative only and is presented for the purpose of enabling those skilled in the art to make and use what is herein disclosed and to teach the best mode of carrying out same. The exclusive rights to all modifications which come within the scope of this disclosure are reserved.

I/We claim:

1. A method for determining senescence based aging or disorder by detection of cells with senescence specific DNA damage markers in a sample comprising one or more cells comprising:
identifying with immunodetection the presence of activated DNA damage response proteins in the sample that are shown to be persistently activated in senescent cells and;
identifying with immunodetection the presence of insignificant activation of DNA damage response proteins in the sample that are shown not to be persistently activated in senescent cells.

2. A method according to claim 1, wherein the senescence based aging or disorder is one or more of age related tissue decline and age related organ decline.

3. A method according to claim 1, wherein the senescence based disorder is one or more of coronary disease, impaired wound healing, inflammation, infection, neoplasia, immune dysfunction, Alzheimer's disease, liver cirrhosis, and immunosenescence.

4. A method according to claim 1, wherein the activation of DNA damage response proteins is determined by determining the kinase activity of kinases selected from the group consisting of ATM/ATR and Chk2.

5. A method according to claim 1, wherein the activated DNA damage response protein is 53BP1.

6. A method according to claim 1, wherein the activated DNA damage response protein is NBS1.

7. A method according to claim 1, wherein the insignificant activation of DNA damage response protein is Chk2.

8. A method according to claim 1, wherein the insignificant activation of DNA damage response protein is P53.

9. A method according to claim 1, further determining that the immunodetection shows insignificant activation of ATM compared to the 53BP1 level.

10. A method for determining senescence based aging or disorder in an organ by detection of cells with senescence specific DNA damage markers in a sample comprising one or more cells from the organ comprising:
identifying with immunodetection the presence of activated DNA damage response proteins in the sample that are shown to be persistently activated in senescent cells and;
identifying with immunodetection the presence of insignificant activation of DNA damage response proteins in the sample that are shown not to be persistently activated in senescent cells.

11. A method according to claim 10, wherein said sample is a tissue biopsy from the organ.

12. A method according to claim 11, wherein said tissue biopsy from said organ is from the liver.

13. A method according to claim 12, wherein said tissue biopsy from the liver is prepared for said immunostaining using standard tissue sectioning and preparation procedures.

14. A method according to claim 13, wherein the activated DNA damage response proteins is selected from the group consisting of 53BP1 and subset of ATM targets that are in DNA damage response foci and the inactivation of DNA damage response proteins is selected from the group consisting of P53,or Chk2.

15. A kit for differentiating senescent and DNA damaged cells comprising:
an immunodetection system for the presence of activated DNA damage response proteins in the sample that are shown to be persistently activated in senescent cells;
an immunodetection system for the presence of inactivation of DNA damage response proteins in the sample that are shown not to be persistently activated in senescent cells;
cell preparation solutions;
blocking agents;
reagents;
a buffer solution and;
a fixing solution.

16. The kit of claim 15, wherein said immunodetection system comprises antibodies.

17. The kit of claim 16, wherein said antibodies are anti-phospho p53, anti-phospho-Chk2 anti-53BP1 and secondary antibodies modified to allow visualization.
